(19) United States
(12) Patent Application Publication
(10) Pub. No.: US 2010/0159506 A1
(43) Pub. Date: Jun. 24, 2010

(54) METHODS AND SYSTEMS FOR GENETIC ANALYSIS OF FETAL NUCLEATED RED BLOOD CELLS

(75) Inventors: Bhairavi Parikh, Palo Alto, CA (US); James Stone, Saratoga, CA (US); Michael D. Brody, Fremont, CA (US); Vivek Balasubramanyam, Belmont, CA (US); Jonathan D. Halderman, Santa Clara, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cellscape Corporation, Mountain View, CA (US)

(21) Appl. No.: 12/509,945

(22) Filed: Jul. 27, 2009

Related U.S. Application Data
(60) Provisional application No. 61/083,784, filed on Jul. 25, 2008.

(51) Int. Cl. C12Q 1/06

(52) U.S. Cl. 435/39

(57) ABSTRACT

Methods for determining genetic status of a fetus from a sample of maternal blood comprise enriching nucleated red blood cells in the sample, including both fetal and maternal nucleated red blood cells. The nucleated red blood cells are then differentiated from all other blood cells in the enriched sample, and the nucleated red blood cells genetically screened to determine the genetic status. The nucleated red blood cells may be differentiated by immobilizing all enriched blood cells on a solid phase and locating the nucleated red blood cells for interrogation. Optionally, the nucleated red blood cells may be sorted and separated from other enriched blood cells in a liquid phase.

### CROSS-REFERENCE TO RELATED APPLICATION

This application claims the benefit of Provisional Application No. 61/083,784 (Attorney Docket No. 027636-000100US), filed on Jul. 25, 2008, the full disclosure of which is incorporated herein by reference.

### BACKGROUND OF THE INVENTION

1. Field of the Invention

The present invention relates generally to genetic analysis and diagnostic techniques. More specifically, the present invention provides methods and systems for determining the genetic status of fetal nucleated red blood cells present in maternal blood circulation.

Non-invasive detection of fetal genetic status chromosomal abnormalities (aneuploidy 21, 18, 13) in the first trimester of pregnancy is a prime objective of prenatal testing. The need for such a test was recently reinforced by the American College of Obstetricians and Gynecologists (ACOG) when it issued new guidelines recognizing the importance of testing all expectant mothers for the risk for genetic disorders (ACOG Practice Bulletin Clinical Management Guidelines For Obstetrician-Gynecologists, Number 77, January 2007). However, the level of risk with current diagnostic tests limits the use of these definitive tests to high-risk populations, generally confined to women over 35 at delivery. Such testing requires a source of fetal cells carrying the fetal genetic material.

Fetal genetic material can be found within circulating fetal cells (CFC's) present in the mother's circulation. CFC's originate in the fetus, cross the placenta, and enter the mother's circulatory system. The most common CFC's are blood cells including trophoblasts, leukocytes, nucleated red blood cells (erythrocytes) and other stem and progenitor cells. Fetal nucleated red bloods (fnRBC's) may be the most useful CFC for fetal genetic analysis. They have a short half life (˜30 days), are relatively abundant in the first-trimester blood, and express easily identifiable hematopoietic plasma membrane antigens. Reliable fnRBC isolation and detection, however, poses significant challenges as approximately one-half of nRBC's in the blood of pregnant women are of maternal rather than fetal origin and many of fnRBC's are undergoing apoptosis and therefore give rise to unstable or fragmented DNA that is not suitable for molecular cytogenetic analysis. In addition, there are only approximately 1-2 circulating fetal nucleated red blood cells (fnRBCs) per milliliter of maternal blood.

Many strategies have been designed to improve the recovery, purity, and yield of fnRBC's from maternal blood but the success rate varies considerably. Negative and/or positive selection may be used to remove unwanted materials through techniques such as density gradient separation, lysis or size based separation. Retention of fnRBC's, however, is problematic. Cells of interest may be enriched using monoclonal antibodies (mAb's) that recognize cell-surface antigens using techniques such as magnetic activated cell sorting or fluorescence activated cell sorting (Ho, et al., Ann Acad Med Singapore (2003) 32:597-604). Efficiency and specificity are limitations of such immunocytochemistry techniques.

As these enrichment methods are non-specific for fnRBC's, methods to distinguish fetal cells from maternal background are employed. Several techniques have been used to differentiate fetal and maternal nRBCs. Cellular morphology distinguishes fetal and maternal nRBC's based on differences in nuclear roundness, nuclear morphology, gamma hemoglobin staining intensity, and peripheral brightness of the stained cytoplasm (Cha, et al., Prenat Diagn (2003) 23:68-73). Embryonic hemoglobin markers are used individually or in combination to determine the transition of hemoglobin from embryonic to fetal to adult chains over time. Markers are either non-specific or decrease rapidly with gestational age as they transition to non-specific markers (Choolani, et. al., Blood (2001) 98: 554-557; and Choolani, et al., Molecular Human Reproduction (2003) 9(4):227-235). X, Y FISH (Fluorescence in-situ hybridization) differentiates male CFC's since a female fetus will not exhibit Y. Other methods for enrichment of fetal cells are described in, e.g., Yamanishi, et al., Expert Rev Mol Diagn (2002) 2(4):303-11.

Currently, the most common procedures used to obtain a fetal sample for prenatal testing include chorionic villus sampling (CVS), amniocentesis, and maternal blood analysis. Amniocentesis and CVS have been the standard in prenatal diagnostic testing for nearly 30 years but are invasive and can pose risk to the developing fetus including a miscarriage rate of 0.25-0.50%. Because amniocentesis is still the most common procedure, expectant mothers often have to wait until 18 weeks into their pregnancy to receive a definitive diagnosis. Current blood tests are available in the first trimester of pregnancy, but are for screening purposes only and are not accurate enough for diagnosis.

It would therefore be of benefit to provide improved methods and systems for obtaining fetal nucleated red blood cells for genetic testing. It would be particularly beneficial to provide methods which would minimize the preparation necessary to determine the genetic status of fetal nucleated red blood cells (fnRBC's) present in maternal circulation. More specifically, it would be desirable to provide methods and protocols which permit genetic analysis of the fnRBC's with a reduced or eliminated need to separate fetal from maternal nucleated red blood cells. At least some of these objectives will be met by the inventions described below.

2. Description of the Background Art

U.S. Pat. No. 7,346,200 describes automated microscopy for detecting fetal cells in a sample and genetically screening the fetal cells. Other patents of interest include U.S. Pat. No. 6,221,607; U.S. Pat. No. 6,136,540; U.S. Pat. No. 5,764,792; and U.S. Pat. No. 5,646,004.

### BRIEF SUMMARY OF THE INVENTION

The present invention provides methods and systems to enable first-trimester, non-invasive, prenatal diagnostics of chromosomal abnormalities. Maternal blood may be obtained as early as 6 to 8 weeks after conception and provides a sample containing fetal nucleated red blood cells (fnRBC) (Wachtel, et al., Clin Genet (2001) 59: 74-79). The concentration of fnRBC's in a maternal sample is approximately 1 in 10⁹ blood cells. To distinguish the nucleated red blood cells (nRBC's) (fetal and maternal) from maternal nucleated red blood cells (mnRBC's), the present invention employs simple sample preparation techniques combined with rapid scanning and/or separation to identify and locate or isolate the target nRBC's, including both fnRBC's and mnRBC's. Conventional molecular diagnostic tests can then be performed on the sample containing both fetal and maternal nucleated red blood cells to detect chromosomal and other genetic abnormalities which are potentially characteristic of the fetus and known not to be present in the mother.

The invention further provides methods by which a sample of maternal blood can be taken with no risk of miscarriage and in which the nucleated red blood cells can be identified and tested for genetic conditions. The sample preparation is usually label-free, and automated image recognition may be used to locate and test the target nucleated RBC's. This invention allows a much higher percentage of pregnant women to access genetic testing by eliminating the miscarriage risk surrounding the procedure. The present methods make testing in higher volume practical by reducing the time in which results are generated from weeks to hours.

In a first aspect, the present invention provides methods for determining genetic status of a fetus. A sample of maternal blood is obtained by conventional techniques, and nucleated red blood cells including both fetal and maternal nucleated red blood cells are enriched in the blood sample. The nucleated red blood cells are then differentiated from all other blood cells in the enriched sample, and at least a portion of the differentiated nucleated red blood cells are screened for genetic status which may be possessed by the fetus but not by the mother. Thus, while the screening will not distinguish between maternal and fetal nucleated red blood cells, if the particular genetic status is located in any of the differentiated nucleated red blood cells, it will indicate that the fetus possesses the genetic status since the mother will be known to be free of that status.

Typically, the genetic status will be a genetic abnormality for which the fetus is being screened. More typically, the genetic abnormality will comprise a chromosomal abnormality, such as aneuploidy, or the like, where it is known that the mother is free from that abnormality. In other cases, the genetic abnormality may comprise a single gene disorder, such as Huntington's disease, cystic fibrosis, and many others. In still other cases, the genetic status may not represent an abnormality. For example, by screening for the presence of a Y chromosome, the gender of the fetus may be determined. If the Y chromosome is present, the fetus is male. If there is no Y chromosome, the fetus is female.

The enrichment step will increase the concentration of fetal and maternal nucleated red blood cells in the blood sample of at least 10-fold over the concentration in the sample, preferably by 40-fold or more, and more preferably by 500-fold or more. The nucleated red blood cells may be enriched in a variety of ways. Most commonly, the non-nucleated red blood cells will be lysed by changing osmolarity, e.g. by exposure to water, glucose, salts; by exposure to ammonium salts; by exposure to acids; by exposure to detergents, or the like. The density of the lysed non-nucleated red blood cells will be quite different than the intact nucleated red blood cells, and the non-nucleated red blood cells may be removed based on this difference in density, typically by density gradient separation or centrifugation. As an alternative to density modification, the non-nucleated red blood cells may be sphered and then separated based on their now smaller size using conventional filtration.

Once the fetal and maternal nucleated red blood cells have been enriched in the maternal blood sample, the nucleated red blood cells will be differentiated from all other blood cells remaining in the enriched sample. Differentiation may be carried out on a solid phase or in a liquid phase. Solid phase differentiation comprises immobilizing all blood cells in the enriched sample, including both the fetal and maternal nucleated red blood cells, on a solid phase substrate. Immobilization may comprise flowing at least a portion of the enriched blood sample over the substrate, usually over a planar surface region of the substrate, and removing liquid components of the sample to affix a layer of the blood cells to the substrate. Optionally, the enriched sample may be drawn into a capillary space formed over the substrate, and the liquid and sample may be removed using an absorbent material. Preferably, the liquid sample is applied under conditions which result in a monolayer of the cells on the substrate. The substrate is preferably a microscope slide or other optically transparent element suitable for optical analysis, as described below.

Alternatively, the enriched blood cells may be immobilized by transferring a predetermined volume, typically from 50 μl to 50 ml, into a small receptacle or well on an optically transparent substrate. The cells are allowed to settle to the bottom of the well or receptacle, typically over a planar region thereof, and the suspension medium may be gelled or solidified to immobilize the cells, using chemicals, temperature, or the like, in a conventional manner. The solidified volume containing the immobilized cells is examined using an optical microscope as described below. Although substrates having planar surfaces that are suitable for microscopic examination are generally preferred, other test protocols may employ other types of solid phase substrates such as microbeads, dipsticks, membranes, and others which are commonly used in biological assays.

Once the enriched blood cells have been immobilized on the substrate, the nucleated blood cells need to be differentiated from all other blood cells. Even though the nucleated red blood cells have been enriched, they still represent only a small portion of the blood cells, typically from 2 in 10⁷ to 1 in 10⁵⁻. The nucleated red blood cells may be differentiated by identifying the locations of the nucleated red blood cells on the substrate by optically scanning the substrate and identifying the nucleated red blood cells based on visual or morphological characteristics. For example, the nucleated red blood cells may be identified using phase contrast microscopy and distinguishing the cells based upon refractive index, and/or the presence of mitochondria, nucleic chromatin, and other features which may be visible. Typically, a single wavelength light, for example, at 650 nm, may be used to effectively distinguish between the nucleated red blood cells and other cells remaining after enrichment. Optionally, a contrast agent may be added, such as DAPI or other nuclear stain, or a cytoplasmic stain, such as eosin Y or benzidine. The increased contrast will be visible in a fluorescent imaging system or an absorption imaging system, and/or a color imaging system.

Once the locations of the nucleated red blood cells on the substrate have been identified, the nucleated red blood cells may be either screened in situ on the substrate or may be transferred from the substrate to a second substrate or liquid phase for genetic analysis. Most commonly, all nucleated red blood cells and other cells immobilized on the substrate will be screened simultaneously by probing the cells using fluorescent in situ hybridization (FISH) or by amplification and probing using polymerase chain reaction (PCR), ligase chain reaction (LCR), or the like.

Screening the cells in situ on the substrate typically comprises recording the coordinates of the locations identified during optical scanning and relying on those coordinates to interrogate the cells at those locations after the cells have been genetically probed or otherwise screened. Recording and storing the coordinates of those locations can be done using an automated microscopy system.

As an alternative to recording the locations of the nucleated red blood cells, the nucleated red blood cells may be tagged with a detectable label which may be used to identify the locations of the nucleated red blood cells after genetic probing of the substrate.

Still further alternatively, the identified nucleated red blood cells could be genetically screened immediately after they are identified on the substrate. The nucleated red blood cells could be probed or otherwise tested while the microscope or other optical system remains focused on the nucleated red blood cells. Generally, however, this latter protocol will not be preferred.

Instead of genetically probing or otherwise testing the nucleated red blood cells while they remain immobilized on the substrate, the nucleated red blood cells may be transferred from the identified locations to another receptacle or substrate for analysis. For example, the transferred nucleated red blood cells could be immobilized on a second substrate for analysis. The advantage of immobilizing them on the second substrate is that the nucleated red blood cells will be present at a much higher percentage than on the first substrate, simplifying the subsequent genetic screening. Alternatively, the immobilized nucleated red blood cells could be collected and suspended in a liquid for liquid phase analysis which will also result in an increased concentration of nucleated red blood cells.

The nucleated red blood cells in the enriched sample may also be differentiated and collected in a liquid phase without prior immobilization and separation. Such sorting may be performed in an automated cell sorting system based on the physical and morphological characteristics of the nucleated red blood cells as discussed above. The assorted nucleated red blood cells in the liquid phase may then be screened in the liquid phase or may be separated and immobilized on a separate substrate for genetic screening and analysis by any of the methods described above for solid phase screening.

Optical scanning of the cells to identify nucleated red blood cells, either on a substrate or in a liquid phase, may be performed by directing a light beam at a wavelength which is strongly absorbed by hemoglobin, where the nucleated red blood cells will absorb light in a ring around the nucleus. Such screening may be performed using contrast phase microscopy, preferably in the presence of a contrast agent. The light may be directed at a multiplicity of wavelengths wherein at least one wavelength is outside the range of hemoglobin absorbance to act as a reference. For example, a first absorptive wavelength may be at 450 nm, a second absorptive wavelength may be at 530 nm, and a third reference wavelength may be in the red or infrared region, where three sequential images may be taken and analyzed to remove extraneous sources of absorbance. Alternatively, the cells could be differentiated based on the appearance of the cell nuclei independent of hemoglobin absorbance. In the latter case, the cells may be scanned with 260 nm light to detect the nuclei.

It is a particular advantage of the present invention that a relatively low number of nucleated red blood cells need to be screened in order to determine the genetic status of the fetus. Typically, the ratio of fetal nucleated red blood cells to maternal nucleated red blood cells will be in the range from about 1:40 to 1:2.5. Thus, by screening at least about 100 nucleated red blood cells, preferably about 300 nucleated red blood cells, the screening of at least some fetal nucleated red blood cells will be assured. A further particular advantage of the present invention is that there is no need to distinguish fetal from maternal nucleated red blood cells.

In a second aspect, the present invention provides methods for identifying an increased risk of abnormal pregnancy in a pregnant female. A sample of maternal blood is provided, nucleated red blood cells in the sample are enriched, and the nucleated red blood cells differentiated from all other red blood cells in the enriched sample. The number of nucleated red blood cells in the enriched sample is then determined, where a number of nucleated red blood cells which is elevated in comparison to a normal number indicates an increased risk of abnormal pregnancy in the pregnant female. As with the screening methods described before, the fetal nucleated red blood cells and the maternal nucleated red blood cells do not need to be distinguished, and the determination may be made during the first trimester of pregnancy. The screening methods are particularly suitable for determining an increased risk of preeclampsia.

### DETAILED DESCRIPTION OF THE INVENTION

### Definitions

The phrase “nucleated red blood cells” or “nRBCs” refer to blood cells that are generally larger and more immature than reticulocytes and mature red blood cells (RBCs). These immature, nucleated stages of the erythrocyte generally occur within the bone marrow. They appear as metarubricytes in small numbers in response to acute blood loss or anemia. Circulating nucleated RBCs can be metarubricytes or younger cells, such as rubricytes.

The phrase “genetic status” may include any chromosomal or single gene status of the fetus which is different from that of the mother. Usually, it will be desirable to screen the fetus for abnormal conditions where it will be apparent that the mother does not possess the condition and therefore would have a different genetic status. Examples of chromosomal abnormalities for which screening may be desired include aneuploidy, of chromosomes 13, 18, 21, X or Y, and the like. Examples of single gene disorders for which screening may be performed include Huntington's disease, cystic fibrosis, and the like. In addition to screening for abnormal conditions, the fetus gender may be determined by screening for the Y chromosome which will necessarily be absent in the mother.

### Sample Preparation and Enrichment

A preliminary separation of red blood cells may be obtained by a single density gradient to separate mononuclear cells, including nucleated red blood cells, from a whole blood sample. Since nRBCs are more dense than white blood cells, it is necessary to use greater density gradients to recover a high yield of nRBCs. Using a 520 mOsm, single density gradient of 1.119 g/ml, a minimum of 4 cells/ml are identified (Kwon, et al., Prenat Diagn (2007) 27(13):1245-50). The sample is then applied to a slide such that the cells are in a monolayer at a density sufficient to view about 1000 cells in a single field. The fixed cells are differentially stained for nuclear and cytoplasmic material using May-Grunwald-Giemsa staining Mavrou, et al., Prenat Diagn (2007) 27:150-153).

### Location and Identification

Identification and location of cells may be based on simple color and intensity discrimination of various cell types after staining Red blood cells will be anuclear. Red and white blood cells, containing a stained nucleus, will be have a dark blue nucleus and a light blue cytoplasm, Nucleated red blood cells will have a bright pink/purple cytoplasm and a dark blue nucleus. Fetal nucleated blood cells need not be separately identified from maternal nucleated blood cells. For example, the fetal cells are not distinguished from maternal cells, e.g., by the labeling of a specific fetal marker(s), e.g., fetal hemoglobin, ε-globin, etc.

An auto-focusing microscope may be used to image areas on a cell plate holding 1000 cell equivalents per image, with a 1000 pixels per cell (a megapixel camera equivalent). The illumination comes from a fiber bundle with a multiplicity of selective wavelengths of light. Images are then sequentially taken, e.g., using wavelengths selected to enhance color enhancement and contrast, including without limitation red and blue wavelengths. Ratiometric techniques, known in the art, are used to discriminate between cell types. Image (pixel) coordinates combined with stage coordinates are used to “locate” the cells on the substrate. The digital microscope also provides the thermocycling and the display for the technician to perform FISH subsequent to location. The successful use of automated auto-focusing microscopes for the detection and analysis of rare cell populations, including fetal nucleated blood cells circulating in maternal blood, has been demonstrated. See, e.g., Oosterwijk, et al., Am J Hum Genet (1998) 63:1783-92; Bajaj, et al., Cytometry (2000) 39:285-94; Merchant and Castleman, Human Reproduction Update (2002) 8(6):509-21. Autofocusing microscopes and accompanying software are known in the art and commercially available. See, e.g., U.S. Pat. Nos. 5,239,170 and 5,790,710; PCT Publ. Nos. WO 2000/075709 and WO 1996/001438. Microscopes with digital autofocus systems are commercially available from, e.g., Olympus America and Oplenic, Hangzhou, China (on the worldwide web at oplenic.com). Imaging software is available from, e.g., Genetix, Hampshire, Great Britain, and Meyer Instruments, Houston Tex.

### Analysis

Currently available techniques for analysis include FISH and standard PCR methods. FISH is the preferred method of analysis as it is FDA cleared and can be applied to cells that are still tethered to the slide, removing the need to extract cells from the plate. Both whole cell techniques and free-DNA techniques require discrimination between fetal in origin genetic material, i.e. fetal specific markers (on the worldwide web at cellbio.dote.hu/angol/description_maygrunwald.pdf; Toeger, et al., Molecular Human Reproduction (1999) 5(12) 1162-1165); and Wataganara et al., Ann N Y Acad Sci. (2004) 1022:90-9). In addition, free DNA techniques are statistical in nature due to the uncertainty of not knowing how many cells the genetic material originated from. Analysis of samples employ an effective method that combines the strengths of both cell-based techniques and free-DNA techniques by analyzing a combined sample, thereby removing the need to determine maternal or fetal origin while retaining the knowledge of the number of cells. For aneuploidy, the method involves looking for a number of chromosomes/number of nucleated cells greater than 2, or by looking for the occurrence of at least one nucleated cell which is positive for aneuploidy (an abnormal number of a specific chromosome).

Thus, a sample of maternal blood may be separated and then applied to a slide. In an exemplary embodiment, separation is accomplished using a density gradient column that retains most mononuclear cells and discards most non-nucleated red blood cells. Nucleated red blood cells (nRBCs)—both maternal and fetal—are identified by computer image processing of visible-light microscopic images. FISH analysis is performed on all nRBCs applied to the slide, e.g., at least about 1, 5, 50, 75, 100, 150, 200, 300, or more, fnRBCs, and statistical techniques are used to infer the existence of certain genetic traits or disorders in the fetus. A positive finding of aneuploidy indicates a chromosomal abnormality. A positive finding of a Y chromosome, indicates that the fetus is male.

The methods find use in the determination of the presence of chromosomal abnormalities (e.g., aneuploidy) or in determining the gender of the fetus. The methods find further application in the prediction of potential hypertensive events that may lead to premature birth (e.g., pre-eclampsia), e.g., by testing for elevated numbers of nucleated red blood cells in relation to a range considered to be normal numbers of nucleated red blood cells (Lana, et al., Am Fam Physician (2004) 70:2317-24; and Mavrou, et al., Prenat Diagn (2007) 27:150-153). Evaluating populations of nucleated red blood cells from the mother can also be used to evaluate the fetus for the presence genetic disorders including Cystic Fibrosis, or for RhD incompatibility using diagnostic methods known in the art (on the worldwide web at americanpregnancy.org/prenataltesting/).

### EXAMPLE

The following examples are offered to illustrate, but not to limit the claimed invention.

Draw whole blood into EDTA or into CPD, CPD-A to prevent coagulation. Preserve a smear of the blood sample. Transfer a sample of blood into a 15 mL tube. If the hematocrit is appreciably higher or lower than 50%, use less or more blood as appropriate to end with a packed red cell volume of ˜3 mL after centrifugation.

The blood is centrifuged to reduce the serum content, to reduce clot formation during the lysis and fixation phase of the procedure. Spin at 2000 g for 10 minutes. Remove the vial from the centrifuge and verify that the volume of the packed red cell layer is approximately 3 mL. The serum and platelets can be discarded to concentrate the target cell population (nucleated RBC's). The nucleated red cells have a density close to WBC's and younger RBC's that are found close to or in the Buffy layer. The Buffy coat layer which separates the packed RBC's from the serum, should not be removed as waste. Using a pipette, remove the top serum layer. Remove as much serum as possible, while making sure to leave the Buffy coat undisturbed. This step requires care.

Transfer the Buffy and red cell layer (along with whatever is left of the serum) into a 50 mL tube. Measure the volume of the packed red cells, and add approximately 6 times that volume of NuFix™ (QCSciences, Richmond, Va.). NuFix lyses the non-nucleated RBC's but stabilizes nucleated cells such as nRBC's and WBC's. The blood cells have to be mixed and resuspended in the NuFix because the cells are denser than the liquid. If the cells are not adequately suspended, the NuFix will not lyse all the RBC's to completion. Gently mix the tube using a back-and-forth motion by hand to suspend the packed red cells. Set the sample upright for 30 minutes (give or take 10 minutes). Spin the NuFix-blood sample at 450 g for 12 minutes. While the sample is spinning, prepare a glycerin-NuFix solution (1:3 glycerin:NuFix by volume). Extract the supernatant from the NuFix-blood sample, leaving 1 ml of solution in the bottom of the centrifuge tube. Mix the remaining supernatant through the pellet to resuspend the cells. Pour 1 mL of glycerin solution into a 15 mL round-bottom tube. Pipette the 1 mL of NuFix-blood solution down the side of the tube so that it forms a layer above the glycerin solution. Spin at 450 g for 6 minutes. Remove the supernatant without disturbing the pellet. Gently mix the remaining supernatant through the pellet to yield approximately 0.5 mL of resuspended cells in solution. Preserve at least one smear of the final sample.

The resuspended cells in solution include nRBC's, other nucleated cells, erythrocyte ghosts, and some platelets. The cell suspension contains the target cell population at higher concentration compared to the starting whole blood sample. Removal of most of the RBCs reduces the number of hemoglobin containing cells that need to be interrogated to identify a nucleated RBC. Reducing the number of RBC's also reduces the volume of cells to be analyzed. A relatively small volume of cells can be more conveniently turned into a relatively small monolayer, several orders of magnitude smaller than a monolayer composed of billions of RBC's, compared to a monolayer composed of millions of nucleated cells.

A capillary fixture for monolayer creation is assembled using two microscope slides (50 mm×75 mm×1 mm) [Premiere Microscope Slides—VWR #48300-309]. The slides are cleaned using soapy water and rinsed with isopropyl alcohol to accelerate drying. The cell immobilization substrate (bottom slide in the capillary) is coated with a cell affixing medium such as Poly-D-Lysine [BD Biosciences, VWR #47743-736]. The top left corner of the cell immobilization slide is marked using a carbide tipped scribe such that the 75 mm edge is parallel to the X axis, the 50 mm edge is parallel to the Y axis, and the Poly-D-Lysine side is facing up. Teflon® tape [McMaster-Carr 76475A41] is used to create 300 um tall standoffs 2.5 mm wide along the short edges of the capillary cap (top slide).

After the coating process is completed and the standoffs are attached to the top slide, the slides are affixed face to face to create a capillary in such a way that the long edges of the slides are coincident, the spacing between the slides is 300 um, and the coated surface of the immobilization slide is toward the inside of the capillary. The internal volume of this capillary cavity is approximately 1 ml.

The cell suspension that results from the enrichment step is diluted to a final volume of 1 ml using Phosphate Buffered Solution [AccuGENE 1X PBS—VWR # 12001-764]. The diluted solution is gently resuspended into the PBS by gently shaking the centrifuge tube. The diluted cell suspension is introduced into the capillary using a pipette.

After the cells have been allowed to settle for approximately 30 minutes, the excess liquid is drawn out of the capillary using an absorbent cloth [Kimwipe—VWR # 21905-026] held against the edge of the capillary opening. After the excess liquid has been removed from the capillary chamber, the cells are allowed to dry in room temperature air for 30 minutes. After drying, the capillary cap (top slide plus Teflon spacers) is removed and the immobilization slide is allowed to air dry at room temperature for another 30 minutes.

After drying, the immobilization slide is placed onto an upright microscope [Olympus BX40 fitted with LUDL MAC2000 controller] with the Poly-D-Lysine coated side facing up, toward the microscope objective. The slide is clipped into place on a mechanical microscope stage capable of moving to and recording accurate XYZ locations [Micos MS-4 for XY, Z is read from the focus control of the LUDL MAC2000]. This microscope is configured to allow imaging of the slide using light transmitted through the cells to be interrogated.

The immobilization slide is aligned by centering the top left, bottom left, and bottom right corners at the center of the field of view, aligned with the center of the microscope reticle. At each of these locations, the XY and focus location of the mechanical positioners is recorded by a computer controlling the motion system. The computer then calculates a motion path to allow digital images to be acquired in such a manner that the complete immobilization slide is imaged. This is accomplished by moving to XY and focus locations that are separated in X and Y by the size of the camera's field of view and stepping through all of these locations until the entire slide has been imaged. At each location, three images are acquired: one using 420 nm transmitted light (blue), one using 520 nm transmitted light (green), and one using 620 nm transmitted light (red). Nucleated red blood cells in each field of view are identified and distinguished from white blood cells based on the absorption ratios of the three wavelengths of light.

Each time a field of view including nucleated red blood cell is located, the XY location is stored in a data file to allow that field of view having the nRBC's to be revisited after the genetic testing has been performed. After the complete slide has been imaged, the slide is processed for genetic testing using a Fluorescent In-Situ Hybridization (FISH) protocol as follows.

The cells are not stained with Giemsa. Store slides with smears or monolayers covered with seal wrap at room temperature. To conserve reagents, isolate the target cells to be probed with a marking pen underneath the slide or monolayer, or use a PAP-Pen directly on the smear or monolayer. The PAP-Pen will provide a barrier so that the reagents will not run across the entire slide. To prevent non-specific binding of the reporter containing the fluorescent tagged antibody to the probe, block the target cells with 10% normal mouse serum in TBST (1.2 (w/v) % Tris, 8.7 (w/v) % NaCl, 0.5% (v/v) Tween-20, 0.1 (w/v) % sodium azide) for 10 min under 740 Torr vacuum chamber at room temperature. Incubate slides for 10 min in 2×SSC (0.15 M NaCl and 0.015 M sodium citrate, pH 7.0) prewarmed to 37° C. in a staining container in a 740 Torr vacuum chamber. Dehydrate sequentially in 70%, 85% and 100% ethanol series, 2 min each at atmospheric pressure. Air dry. Redraw the circle with a PAP-Pen. Vysis® DNA probes are prepared by combining 7 μl buffer (comes together with Vysis probes), 1 μl dH2O, 1 μl of each probe (for example CEP6, Spectrum Green probe and CEP17 Spectrum Orange probe). Centrifuge 1-3 seconds, vortex, and recentrifuge. Heat for 5 min. at 73° C. in a water bath to denature. Use immediately (or keep for a short while longer at 73° C. if required).

### Slide Preparation

Place denaturant solution (70% formamide/2×SSC) in 73° C. water bath inside staining jar. Denature slide for 5 min. Dehydrate in 70%, 85% and 100% ethanol for 2 min. each. Air dry. Apply 10 μl denatured probe and cover with a cover glass. Mark hybridizing area on the slide using a diamond scribe or Pap-pen. Seal carefully with rubber cement. Place slides in a pre-warmed humidified box (wrapped in metal foil to protect against light) and incubate 2 hours in a 740 ton vacuum at 42° C.

### Wash

Place 0.4×SSC/0.3% NP-40 in a 73° C. water bath. Remove cover glass and immediately place into wash tank with 0.4×SSC/0.3% NP-40. Leave all slides in staining jar for 2 min. Place slides in 2×SSC/0.1% NP-40 at room temperature 1 min. Air dry slides in darkness. Apply 20 μl of Vectashield with DAPI solution to the target area and put on cover glass (make sure it covers hybridized area). Examine slides on a fluorescence microscope.

### Solutions

2×SSC (pH to 7.0)
0.4×SSC/0.3% NP-40 (pH 7.0-7.5)
2×SSC/0.1% NP-40 (pH 7.0-7.5)
Denaturant solution: 49 ml Formamide, 7 ml 20×SSC, 14 ml dH2O, pH to 7.0-8.0, store at 4° C.
Ethanol Wash Solutions: 70%, 85%, 100%

After the cells have been processed according to the FISH protocol and the target nRBC's labeled, the immobilization slide is placed onto an upright microscope [Olympus BX40 fitted with LUDL MAC2000 controller] with the Poly-D-Lysine coated side facing up, toward the microscope objective. The slide is clipped into place on a mechanical microscope stage capable of moving to and recording accurate XYZ locations [Micos MS-4 for XY, Z is read from the focus control of the LUDL MAC2000]. This microscope is configured to allow imaging of the slide using coaxial fluorescent imaging.

The immobilization slide is aligned by centering the top left, bottom left, and bottom right corners at the center of the field of view, aligned with the center of the microscope reticle. At all of the locations previously determined to be nucleated red blood cells, analysis of the FISH results is performed based on the specific FISH protocol that is followed. The results of many nucleated red blood cell FISH analyses are combined using statistical algorithms to improve the confidence in the final data that is reported.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for determining genetic status of a fetus, said method comprising;
providing a sample of maternal blood;
enriching nucleated red blood cells including both fetal and maternal nucleated red blood cells in the maternal blood sample;
differentiating the nucleated red blood cells from all other blood cells in the enriched sample; and
screening at least a portion of the differentiated nucleated red blood cells for a genetic status which may be possessed by the fetus but not by the mother.

2. The method of claim 1, wherein the genetic status comprises a genetic .abnormality unique to the fetus.

3. The method of claim 2, wherein the genetic abnormality comprises a chromosomal abnormality.

4. The method of claim 3, wherein the chromosomal abnormality is aneuploidy.

5. The method of claim 2, wherein the genetic abnormality comprises a single gene disorder.

6. The method of claim 1, wherein the genetic status comprises the presence of a Y chromosome which determines gender of the fetus.

7. The method of claim 1, wherein enriching comprises increasing the concentration of fetal and maternal nucleated red blood cells in the enriched sample by at least 10 fold over the concentration in the sample.

8. The method of claim 1, wherein enriching comprises selectively modifying the density of the non-nucleated red blood cells and separating non-nucleated red blood cells from nucleated red blood cells based on density.

9. The method of claim 8, wherein modifying the density comprises selectively lysing non-nucleated red blood cells.

10. The method of claim 8, wherein modifying the density of the non-nucleated red blood cells comprises increasing the density with a density modification agent.

11. The method of claim 8, wherein the non-nucleated red blood cells are separated in a density gradient.

12. The method of claim 1, wherein enriching comprises sphering the non-nucleated red blood cells and filtering them from the sample based on shape.

13. The method of claim 1, wherein differentiating comprises immobilizing blood cells including fetal and maternal nucleated red blood cells from the enriched sample on a substrate.

14. The method of claim 13, wherein immobilizing comprises flowing at least a portion of the blood sample over a substrate and removing liquid components of the blood to affix a layer of blood cells to the substrate.

15. The method of claim 14, wherein flowing comprises drawing the blood sample into a capillary space formed over the substance.

16. The method of claim 13, further comprising identifying two locations of the immobilized nucleated red blood cells on the substrate.

17. The method of claim 16, wherein identifying the locations comprises optically scanning the substrate and identifying nucleated red blood cells based on visual characteristics.

18. The method of claim 17, further comprising recording the coordinates of those locations having identified nucleated red blood cells so that said locations can be subsequently interrogated based on the coordinates.

19. The method of claim 17, wherein further comprising labeling the identified nucleated red blood cells with a detectable label so that the nucleated red blood cells may be subsequently screened based on presence of the label without screening other immobilized blood cells.

20. The method of claim 17, wherein the identified nucleated red blood cells are genetically screened substantially immediately after they are identified.

21. The method of claim 16, further comprising transferring nucleated red blood cells from the identified locations to another receptacle for analysis.

22. The method of claim 20, wherein the transferred nucleated red blood cells are immobilized on a second substrate for analysis.

23. The method of claim 20, wherein the transferred nucleated red blood cells are collected and suspended in a liquid for analysis.

24. The method of claim 1, wherein differentiating comprises sorting the enriched cells to separate nucleated red blood cells from all other blood cells and collecting the separated nucleated red blood cells in a liquid phase.

25. The method of claim 24, further comprising immobilizing the separated nucleated red blood cells on a second substrate, wherein screening is performed on the immobilized nucleated red blood cells.

26. The method of claim 24, wherein screening is performed on the nucleated red blood cells in the liquid phase.

27. The method of claim 17, wherein optically scanning comprises directing a light beam at a wavelength which is strongly absorbed by hemoglobin at a plurality of immobilized blood cells, wherein those blood cells which absorb said light in a ring around a nucleus are determined to be nucleated red blood cells.

28. The method of claim 27, further comprising adding a contrast agent to the cells to increase contrast between the nucleus and the hemoglobin.

29. The method of claim 27, wherein light absorbance is determined by phase contrast microscopy.

30. The method of claim 27, further comprising directing light at multiple wavelengths wherein at least one wavelength is outside the range of hemoglobin absorbance to act as a reference.

31. The method of claim 30, wherein a first absorptive wavelength is at 415 nm, a second absorptive wavelength is at 530 nm, and a third reference wavelength is in the red or infrared region, wherein three sequential images are taken and analyzed to remove extraneous sources of absorbance.

32. The method of claim 27, further comprising detecting cell nuclei independent of hemoglobin absorbance.

33. The method of claim 32, wherein the cells are scanned with 260 nm light to detect nuclei.

34. The method of claim 14, wherein screening comprises probing all cells immobilized on the substrate simultaneously.

35. The method of claim 1, wherein differentiating comprises separating the fetal and maternal nucleated red blood cells from other blood cells in the sample to produce a liquid phase consisting primarily of nucleated red blood cells.

36. The method of claim 35, wherein screening comprises probing all cells in the liquid phase fraction simultaneously.

37. The method of claim 35, further comprising transferring at least some of the nucleated red blood cells from the liquid phase to a substrate and immobilizing said transferred cells on the substrate.

38. The method of claim 1, wherein the genetic status is determined by fluorescent in situ hybridization (FISH).

39. The method of claim 30, wherein the hybridization is performed under a partial vacuum.

40. The method of claim 1, wherein the genetic status is determined by polymerase/ligase chain reaction (PCR/LCR).

41. The method of claim 1, wherein the genetic status of at least about 100 nucleated red blood cells are determined.

42. The method of claim 1, wherein the fetal nucleated red blood cells and the maternal nucleated red blood cells are not distinguished.

43. The method of claim 1, wherein the chromosomal status is determined during the first trimester of pregnancy.

44. A method for identifying an increased risk of abnormal pregnancy in a pregnant female, said method comprising;
providing a sample of maternal blood;
enriching nucleated red blood cells including both fetal and maternal nucleated red blood cells in the maternal blood sample;
differentiating the nucleated red blood cells from all other blood cells in the enriched sample; and
determining the number of nucleated red blood cells in the sample, wherein an elevated number of nucleated red blood cells in comparison to a threshold number indicates an increased risk of abnormal pregnancy in the pregnant female.

45. The method of claim 44, wherein the fetal nucleated red blood cells and the maternal nucleated red blood cells are not distinguished.

46. The method of claim 44, wherein the number of nucleated red blood cells is determined during the first trimester of pregnancy.

47. The method of claim 44, wherein the abnormal pregnancy is preeclampsia.
